(19) United States
(12) Patent Application Publication
(10) Pub. No.: US 2024/0237949 A1
(43) Pub. Date: Jul. 18, 2024

(54) IMD WITH CERAMIC HOUSING

(71) Applicant: Cardiac Pacemakers, Inc., St. Paul, MN (US)

(72) Inventors: Jean M. Bobgan, Maple Grove, MN (US); Keith R. Maile, New Brighton, MN (US); Daniel Joseph Landherr, Wyoming, MN (US); Derek John Boettger, Woodbury, MN (US)

(21) Appl. No.: 18/413,939

(22) Filed: Jan. 16, 2024

Related U.S. Application Data
(60) Provisional application No. 63/439,697, filed on Jan. 18, 2023.

(51) Int. Cl. A61B 5/00

(52) U.S. Cl. CPC A61B 5/6867; A61B 2560/0406; A61B 2560/0468

(57) ABSTRACT

A medical device includes a first housing section with a first cavity and including a ceramic material, a second housing section coupled to the first housing section and including a second cavity, a first circuit board section positioned within the first cavity, a second circuit board section positioned within the second cavity, and an antenna coupled to the first circuit board section.

### CROSS REFERENCE TO RELATED APPLICATION

This application claims priority to Provisional Application No. 63/439,697, filed Jan. 18, 2023, all of which are herein incorporated by reference in their entirety.

### TECHNICAL FIELD

Instances of the present disclosure relate to medical devices and systems for sensing physiological parameters and/or delivering therapy. More specifically, instances of the disclosure relate to implantable medical devices with one or more housing components that comprise a ceramic material.

### BACKGROUND

Certain implantable medical devices can be configured to sense physiological parameters and/or provide therapy and may include one or more electrodes for performing aspects of these functions. Further, certain implantable medical devices can include an antenna that is communicatively coupled to device external to the patient.

### SUMMARY

In Example 1, a medical device includes a first housing section with a first cavity and including a ceramic material, a second housing section coupled to the first housing section and including a second cavity, a first circuit board section positioned within the first cavity, a second circuit board section positioned within the second cavity, and an antenna coupled to the first circuit board section.

In Example 2, the medical device of Example 1, wherein the antenna is embedded in the first circuit board section.

In Example 3, the medical device of Examples 1 or 2, wherein the first circuit board section and the second circuit board section comprise a rigid circuit board or a flexible circuit.

In Example 4, the medical device of any of Examples 1-3, wherein the first circuit board section is a flexible circuit board and the second circuit board section is a rigid circuit board.

In Example 5, the medical device of any of Examples 1-4, wherein the first housing section includes a distal end and a proximal end, wherein the proximal end includes a single opening through which separate conductors extend through.

In Example 6, the medical device of Example 5, wherein the separate conductors are separate traces embedded within the first circuit board section and the second circuit board section.

In Example 7, the medical device of any of Examples 1-6, wherein the first circuit board section and the second circuit board section comprise a single, continuous circuit board that extends through the first cavity and the second cavity.

In Example 8, the medical device of any of Examples 1-6, wherein the first circuit board section and the section circuit board section are separate circuit boards that are electrically coupled to each other.

In Example 9, the medical device of any of Examples 1-8, wherein the medical device does not include an electrical feed-through positioned between the first housing section and the second housing section.

In Example 10, the medical device of Example 1, further including a first electrode coupled to the first housing section, a third housing section housing a battery, and a second electrode coupled to the third housing section.

In Example 11, the medical device of Example 10, wherein a pin or spring contact is electrically and mechanically coupled between the first electrode and the first circuit board section.

In Example 12, the medical device of Example 11, wherein the pin or the spring contact extends through an aperture in the first housing section.

In Example 13, the medical device of any of the preceding Examples, further including a third housing section coupled between the first housing section and the second housing section.

In Example 14, the medical device of any of the preceding Examples, wherein the antenna is positioned within the first cavity.

In Example 15, the medical device of any of the preceding claims, wherein the ceramic material comprises zirconia.

In Example 16, medical device includes a first housing section including a first cavity and comprising a ceramic material, a second housing section coupled to the first housing section and including a second cavity, a first circuit board section positioned within the first cavity, a second circuit board section positioned within the second cavity, and an antenna coupled to the first circuit board section and positioned within the medical device to transmit signals through the ceramic material.

In Example 17, the medical device of Example 16, wherein the first circuit board section and the second circuit board section comprise a rigid circuit board or a flexible circuit.

In Example 18, the medical device of Example 16, wherein the first circuit board section is a flexible circuit board and the second circuit board section is a rigid circuit board.

In Example 19, the medical device of Example 16, wherein the first circuit board section and the second circuit board section form a single, continuous circuit board that extends through the first cavity and the second cavity.

In Example 20, the medical device of Example 16, wherein the first circuit board section and the section circuit board section are separate circuit boards that are electrically coupled to each other.

In Example 21, the medical device of Example 16, wherein the medical device does not include an electrical feed-through positioned between the first housing section and the second housing section.

In Example 22, the medical device of Example 21, wherein separate traces embedded within the first circuit board section and the second circuit board section transmit electrical signals from the first housing section to the second housing section.

In Example 23, the medical device of Example 16, further comprising: a first electrode coupled to the first housing section, a third housing section housing a battery, and a second electrode coupled to the third housing section.

In Example 24, the medical device of Example 23, wherein a pin or spring contact is electrically and mechanically coupled between the first electrode and the first circuit board section.

In Example 25, the medical device of Example 24, wherein the pin or the spring contact extends through an aperture in the first housing section.

In Example 26, the medical device of Example 25, wherein first electrode or the first pin hermetically seals the aperture.

In Example 27, the medical device of Example 16, further comprising: a third housing section coupled between the first housing section and the second housing section, wherein the third housing section is brazed to the first housing section.

In Example 28, the medical device of Example 27, wherein the third housing section is welded to the second housing section.

In Example 29, the medical device of Example 16, wherein the antenna is positioned within the first cavity.

In Example 30, the medical device of Example 16, wherein the ceramic material comprises zirconia.

In Example 31, the medical device of Example 29, wherein the first housing section comprises alumina.

In Example 32, a method for manufacturing an implantable medical device (IMD) includes positioning a first circuit board section in a first cavity formed by a first housing section of an outer housing for the IMD, positioning an antenna in the first housing section and coupling the antenna to traces in the first circuit board, and positioning a second circuit board section in a second cavity formed by a second housing section of the outer housing for the IMD. the first housing section comprises a ceramic material.

In Example 33, the method of Example 32, further comprising: brazing the first housing section to a third housing section and welding the second housing section to the third housing section.

In Example 34, the method of Example 32, further comprising: hermetically sealing an aperture in the first housing section with one or more components of an electrode assembly.

In Example 35, the method of Example 34, further comprising: inserting a conductive pin through the aperture.

While multiple instances are disclosed, still other instances of the present disclosure will become apparent to those skilled in the art from the following detailed description, which shows and describes illustrative instances of the disclosure. Accordingly, the drawings and detailed description are to be regarded as illustrative in nature and not restrictive.

### BRIEF DESCRIPTION OF THE DRAWINGS

FIG. 1 is a schematic illustration depicting a patient monitoring system, in accordance with certain instances of the present disclosure.

FIG. 2 is a side view of an implantable medical device, in accordance with certain instances of the present disclosure.

FIGS. 3 and 4 are partially exploded views of the medical device of FIG. 2, in accordance with certain instances of the present disclosure.

FIG. 5 is a partial cutaway view of the medical device of FIG. 2, in accordance with certain instances of the present disclosure.

FIG. 6 is a partially exploded view of a medical device, in accordance with certain instances of the present disclosure.

FIG. 7 is a partial cutaway view of the medical device of FIG. 6, in accordance with certain instances of the present disclosure.

FIG. 8 is a perspective view of part of the medical device of FIG. 6, in accordance with certain instances of the present disclosure.

FIGS. 9 and 10 show different instances of electrical connections of an electrode of an outer housing section for use with medical devices, in accordance with certain instances of the present disclosure.

FIG. 11 depicts a block diagram of a method of manufacturing the medical devices described herein, in accordance with certain instances of the present disclosure.

While the disclosed subject matter is amenable to various modifications and alternative forms, specific instances have been shown by way of example in the drawings and are described in detail below. The intention, however, is not to limit the disclosed subject matter to the particular instances described. On the contrary, the disclosed subject matter is intended to cover all modifications, equivalents, and alternatives falling within the scope of the disclosed subject matter as defined by the appended claims.

### DETAILED DESCRIPTION

Ceramic materials are useful in many applications including medical device applications because of their biocompatibility in addition to benefits such as pass-through signal transmission (e.g., electromagnetic signal transparency), structural integrity (e.g., corrosion resistance, structural strength), and/or ability to be hermetically sealed. Such materials are useful for various components in medical devices such as sensing devices and therapy devices (e.g., pacemakers, defibrillators).

Certain instances of the present disclosure utilize a ceramic material for one or more sections of a housing for an implantable medical device. Additionally or alternatively, certain instances of the present disclosure utilize a housing design that reduces—or eliminates—certain electrical feed-through assemblies.

### System

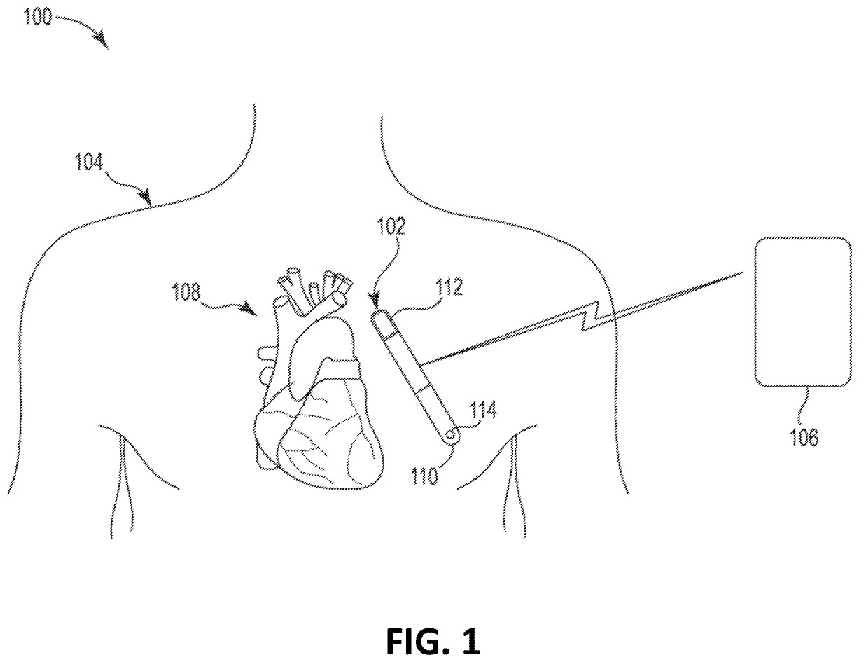

FIG. 1 is a schematic illustration of a system 100 including an implantable medical device (IMD) 102 implanted within a patient's body 104 and configured to communicate with a receiving device 106. Components of the IMD 102 such as sections of the outer housing can comprise a ceramic material.

The IMD 102 may be implanted subcutaneously within an implantation location or pocket in the patient's chest or abdomen and may be configured to monitor (e.g., sense and/or record) physiological parameters associated with the patient's heart 108. The IMD 102 may be an implantable cardiac monitor (e.g., an implantable diagnostic monitor, an implantable loop recorder) configured to record physiological parameters such as, for example, one or more cardiac activation signals, heart sounds, blood pressure measurements, oxygen saturations. Further, the IMD 102 may be configured to monitor physiological parameters that may include one or more signals indicative of a patient's physical activity level and/or metabolic level, such as an acceleration signal.

For purposes of illustration, and not of limitation, various instances of devices that may be used to record physiological parameters in accordance with the present disclosure are described herein in the context of IMDs that may be implanted under the skin in the chest region of a patient. However, the IMD 102 may include any type of IMD, any number of different components of an implantable system, and/or the like having a housing and being configured to be implanted in a patient's body 104. For example, the IMD 102 may include a control device, a monitoring device, a pacemaker, an implantable cardioverter defibrillator (ICD), a cardiac resynchronization therapy (CRT) device and/or the like, and may be an implantable medical device known in the art or later developed, for providing therapy and/or diagnostic data about the patient's body. In various instances, the IMD 102 may include both defibrillation and pacing/CRT capabilities (e.g., a CRT-D device).

As shown, the IMD 102 may include a housing 110 having two electrodes 112 and 114 coupled thereto. The IMD 102 may be configured to sense physiological parameters and record the physiological parameters. For example, the IMD 102 may be configured to activate (e.g., periodically, continuously, upon detection of an event, and/or the like), record a specified amount of data (e.g., physiological parameters) in a memory, and communicate that recorded data to the receiving device 106. The recording device 106 may be, for example, a programmer, controller, patient monitoring system, and/or the like. Although illustrated in FIG. 1 as an external device, the receiving device 106 may include an implantable device configured to communicate with the IMD 102 that may, for example, be a control device, another monitoring device, a pacemaker, an ICD, a CRT device, and/or the like.

The IMD 102 and the receiving device 106 may communicate through a wireless link. For example, as will be described in more detail below, the IMD 102 can include an antenna, which transmits and/or receives signals from the receiving device 106. The IMD 102 and the receiving device 106 may be communicatively coupled through a short-range communications link, such as Bluetooth, IEEE 802.11, and/or a proprietary wireless protocol. The communications link may facilitate uni-directional and/or bi-directional communication between the IMD 102 and the receiving device 106. Data and/or control signals may be transmitted between the IMD 102 and the receiving device 106 to coordinate the functions of the IMD 102 and/or the receiving device 106. The patient data may be downloaded from one or more of the IMD 102 and the receiving device 106 periodically or on command. The physician and/or the patient may communicate with the IMD 102 and the receiving device 106, for example, to acquire patient data or to initiate, terminate, or modify recording and/or therapy.

### Medical Device

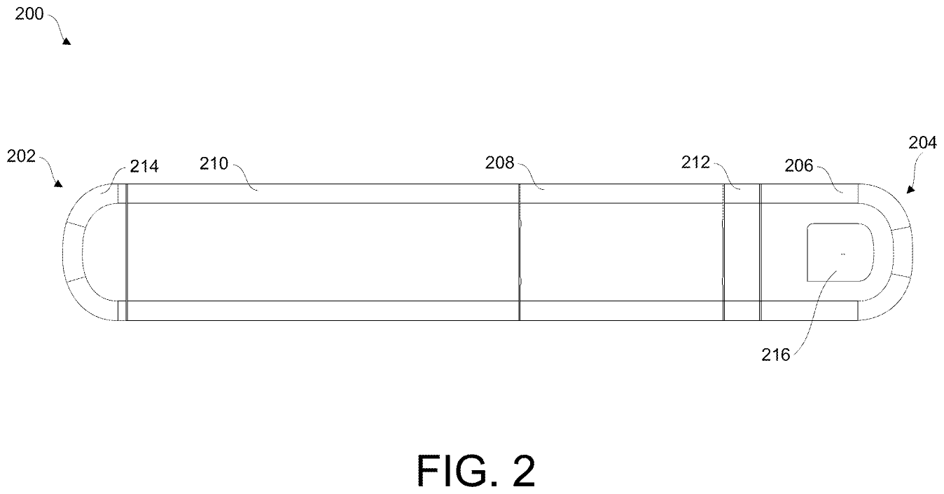

FIG. 2 is a side view of a medical device 200 (hereinafter “IMD 200” for brevity). The IMD 200 may be, or may be similar to, the IMD 102 depicted in FIG. 1 and may be used in the system 100 of FIG. 1.

The IMD 200 includes an external housing that extends between a first end 202 and a second end 204. In the example of FIG. 2, the IMD 200 includes a first housing section 206, a second housing section 208, a third housing section 210, a fourth housing section 212, a first electrode 214, and a second electrode 216. Each of the housing sections can be separate components that are assembled together during manufacturing to create the external housing of the IMD 200. When assembled together, the housing sections can create a hermetically sealed enclosure. Although four separate housing sections are shown in FIG. 2, additional or fewer separate sections can be used to create the IMD 200. As will be described in more detail below, one or more of the housing sections can comprise a ceramic material.

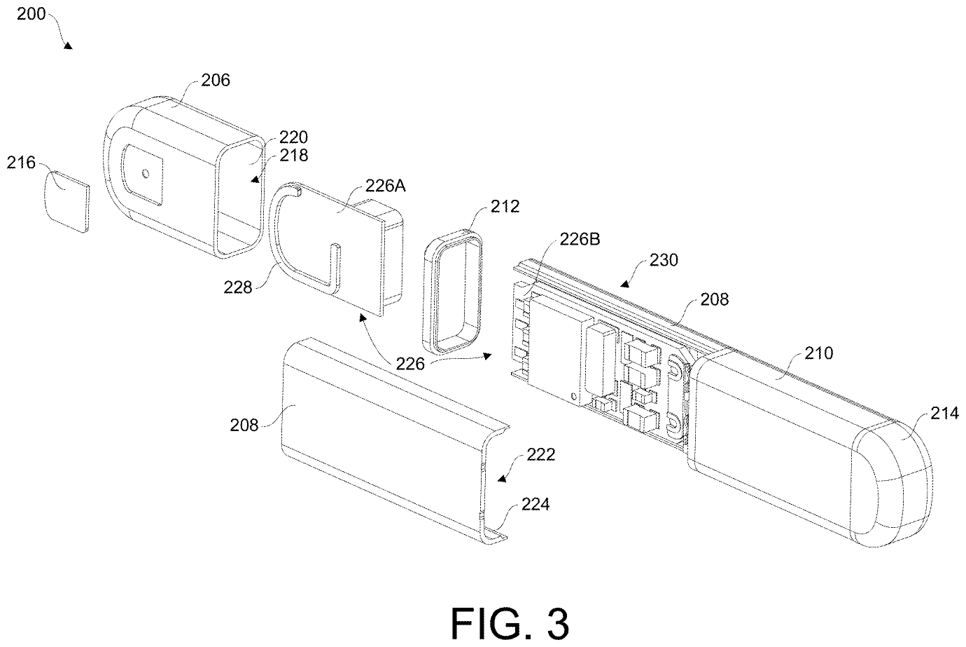

FIG. 3 shows a partially exploded view of the IMD 200. The first housing section 206 includes a first cavity 218 that is defined by one or more interior surfaces 220 of first housing section 206. The second electrode 216 is coupled to the first housing section 206. In certain instances, the first housing section 206 comprises a ceramic material.

The second housing section 208 includes a second cavity 222 that is defined by one or more interior surfaces 224 of second housing section 208. In FIG. 2, the second housing section 208 is shown as being comprised of multiple housing components. For example, the second housing section 208 (as with other housing sections) can be assembled from multiple components (e.g., welded together) to create its portion of the external housing of the IMD 200. In certain instances, the second housing section 208 comprises a metal material such as titanium. In other instances, the second housing section 208 comprises a ceramic material.

The third housing section 210 can comprise a battery assembly (which may include one or more batteries). The exterior of the battery assembly can form the third housing section 210 in which one or more batteries (e.g., rechargeable battery cells) are positioned. The first electrode 214 is disposed at an end of the third housing section 210. In certain instances, the first electrode 214 is integrated with the battery assembly.

The fourth housing section 212 can function as an interface or coupler between the first housing section 206 and the second housing section 208. For example, the fourth housing section 212 can be used to couple the first housing section 206 to the second housing section 208. More specifically, in instances where the fourth housing section 212 comprises a metal such as titanium, the fourth housing section 212 can be assembled to the second housing section 208 via welding (e.g., laser welding). And, in instances where the first housing section 206 comprises a ceramic, the fourth housing section 212 can be brazed to the first housing section 206. In such instances, the fourth housing section 212 can be coupled between the first housing section 206 and the second housing section 208. As shown in FIG. 3, the fourth housing section 212 can be shaped as a continuous ring with an opening therethrough. Further, the fourth housing section 212 can include joint features such as one or more thinned sections or flange sections such that connecting the fourth housing section 212 to the other sections (e.g., via welding and/or brazing) can be accomplished. For example, portions of the other housing sections can overlap with the thinned or flanged sections of the fourth housing section 212 to provide overlapping surface area.

FIG. 3 shows a circuit board 226 with a first circuit board section 226A and a second circuit board section 226B. For purposes of illustrating the various components of the IMD 200 in an exploded view, the first circuit board section 226A and the second circuit board section 226B are shown as two separate components, but the two sections can form a single circuit board. The first circuit board section 226A and the second circuit board section 226B can comprise a rigid circuit board or a flexible circuit (e.g., a flexible circuit comprising polyimide). In instances where the first circuit board section 226A and the section circuit board section 226B are separate components, one section can comprise a rigid circuit board and the other section can comprise a flex circuit. Further, the two sections can be electrically coupled to each other.

When the IMD 200 is assembled, the first circuit board section 226A is positioned within the first cavity 218 and the second circuit board section 226B is positioned within the second cavity 222. Portions of either or both of the first circuit board section 226A and the second circuit board section 226B can extend through the fourth housing section 212 when the IMD 200 is assembled. As such, in instances with a single, continuous circuit board, the circuit board 226 can extend within the first cavity 218 and the second cavity 222 and through the opening of the fourth housing section 212.

An antenna 228 is positioned within the first cavity 218 and coupled to the circuit board 226 such as to the first circuit board section 226A. As one example, the antenna 228 can be embedded within the first circuit board section 226A. In this example, the antenna 228 can be formed by a conductive trace in the first circuit board section 226A. As another example, the antenna 228 can be formed on the interior surface 220 of the first housing section 206 and electrically coupled (e.g., directly coupled or indirectly coupled) to a conductive trace in the first circuit board section 226A. In this example, a portion of the interior surface 220 can be metalized if the interior surface 220 is otherwise a ceramic material. As another example, the antenna 228 can be embedded in the first housing section 206 and electrically coupled to a conductive trace in the first circuit board section 226A.

Various other electrical components 230 can be coupled to the circuit board 226 such as on the second circuit board section 226B. The electrical components 230 can include one or more integrated circuits (e.g., application specific integrated circuits, field-programmable gate arrays) programmed to perform functions such as sensing, processing, and/or communication functions of the IMD 200. For example, the electrical components 230 can include one or more processors (e.g., microprocessors) coupled to memory with instructions (e.g., in the form of firmware, and/or software) for performing functions of the IMD 200.

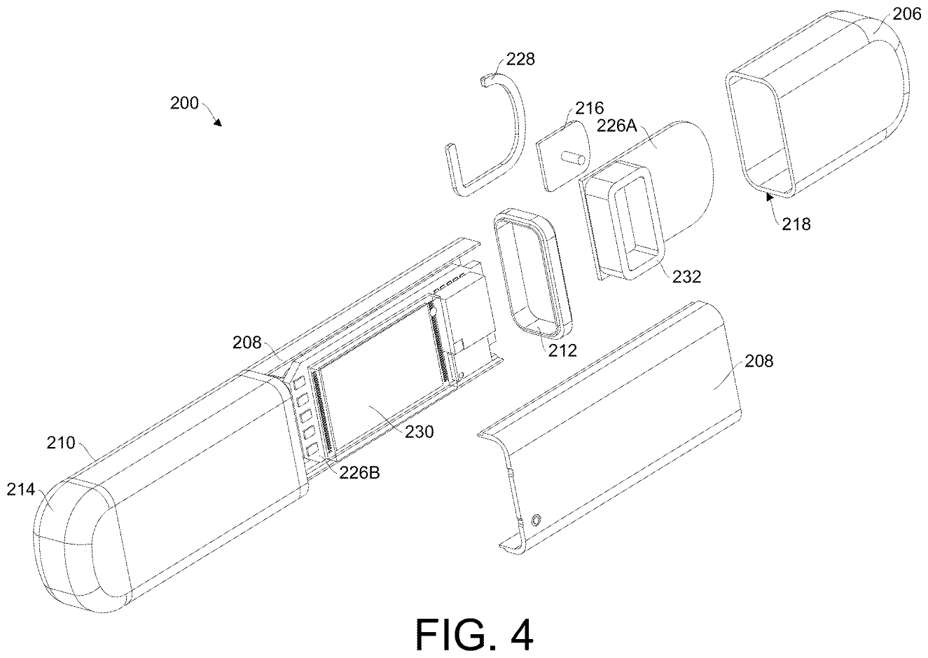

FIG. 4 shows another view of the IMD 200. As shown in FIG. 4, in certain instances, a receiver coil 232 is coupled to the circuit board 226 such as the first circuit board section 226A. The receiver coil 232 is arranged to receive external signals (e.g., electromagnetic signals) that induce a current in the receiver coil 232 such that the current can recharge batteries in the battery assembly. When the IMD 200 is assembled, the receiver coil 232 is positioned within the first cavity 218.

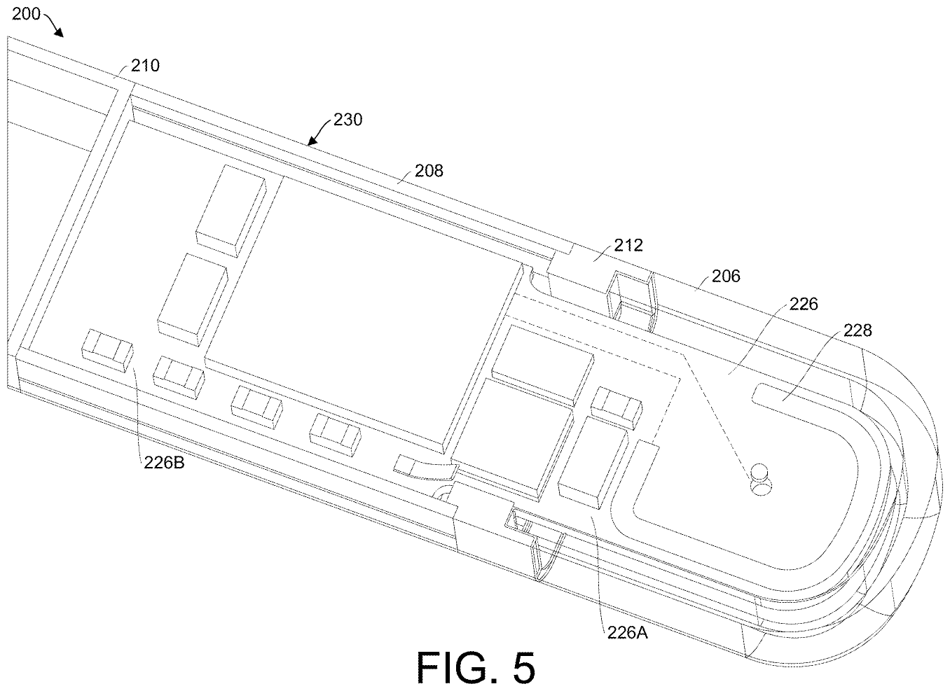

FIG. 5 shows a partial cutaway view of the IMD 200 after being assembled. As shown in FIG. 5, the circuit board 226 extends through the interior of the first housing section 206, the second housing section 208, and the fourth housing section 212. As such, the IMD 200 does not require an electrical feed-through assembly to electrically couple items positioned in the first housing section 206 (such as the antenna 228) to items positioned in the second housing section 208 (such as the electrical components 230 like processors). Instead, conductors (e.g., electrical traces) within the circuit board 226 can electrically couple items in the first housing section 206 to items positioned in the second housing section 208. As such, multiple separate conductors (shown in dotted lines in FIG. 5) can extend through a single opening at the proximal end in the first housing section 206.

By not requiring a separate electrical feed-through assembly to pass signals from one housing section to another, the number of components and/or number of manufacturing processes for the IMD 200 can be reduced. Further, the overall size (e.g., length) of the IMD 200 can be reduced by removing the need for an electrical feed-through assembly between housing sections. Additionally or alternatively, a larger battery can be used without necessarily increasing the overall length of the IMD 200 relative to IMDs that require an electrical feed-through assembly between housing sections. A larger battery can increase the overall battery life of the IMD 200.

As noted above, certain housing sections can comprise a ceramic material—which can be biocompatible, used for providing a hermetic seal, and/or can provide pass-through signal transmission (e.g., electromagnetic signal transparency). The housing sections that surround the antenna or other signal transmission components can comprise a ceramic material. For example, if the IMD 200 included an antenna positioned within the second housing section 208—rather than the first housing section 206—the second housing section 208 could comprise a ceramic material to provide pass-through signal transmission for the antenna.

The one or more ceramic housing sections can include a substrate or base layer comprising a ceramic material such as zirconia-based ceramics. Zirconia-based ceramic materials include, for example, zirconia, stabilized zirconia, partially stabilized zirconia, tetragonal zirconia, magnesia stabilized zirconia, ceria-stabilized zirconia, yttria stabilized zirconia (“YSZ”) such as 3Y-TZP, and calcia stabilized zirconia, as well as alumina (e.g., zirconia toughened alumina (ZTA), ATZ), and titania, and the like. In some instances, the base substrate 602 may include from 5% to about 99.9% zirconia, or from about 5% to about 95% zirconia, or from about 10% to about 93% zirconia, or from about 15% to about 85% zirconia, or from about 20% to about 80% zirconia, or from about 25% to about 75% zirconia, or a percentage of zirconia encompassed within these ranges. The base substrate 602 may also be a mixture of alumina and zirconia, containing about 90% alumina and about 10% zirconia, or about 75% alumina and about 25% zirconia, or about 20% alumina and about 80% zirconia, or about 0.3% alumina and about 93% zirconia. In an exemplary instance, the base substrate 602 comprises YSZ.

As also noted above, when the first housing section 206 comprises a ceramic material, the first housing section 206 and the fourth housing section 212 can be brazed to couple the sections together. In such instances, the portion of the first housing section 206 used for brazing (e.g., the brazing interface portion) can be metallized to provide a bonding surface for the brazing material. Metallizing may involve depositing a tie layer (e.g., layer comprising titanium, chromium, or vanadium) directly onto the ceramic substrate in order to facilitate metallization of the ceramic substrate.

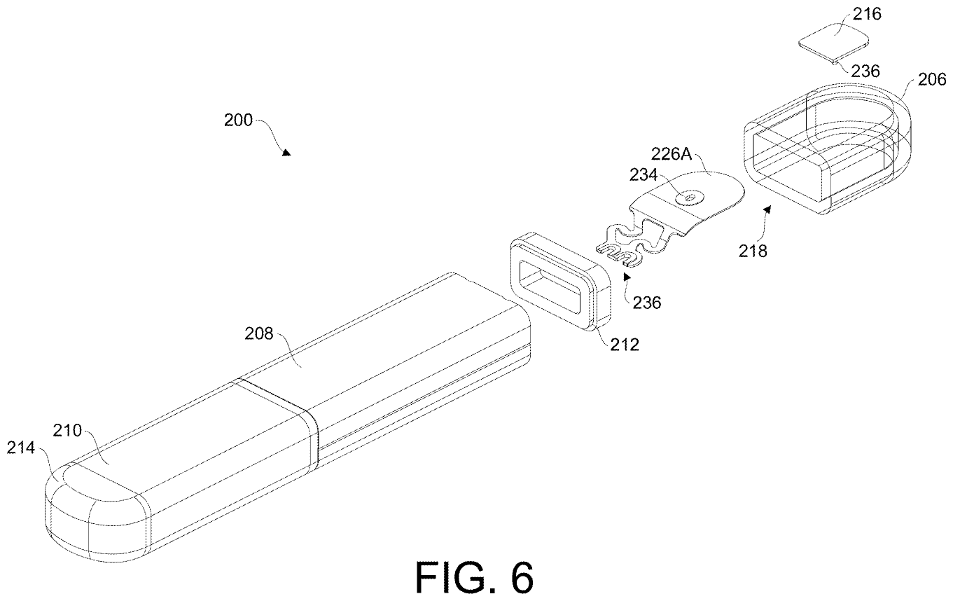

FIGS. 6-8 show another example approach for electrically coupling items positioned in the first housing section 206 to items positioned in the second housing section 208. In this example, the first circuit board section 226A (positioned in the first cavity 218) and the second circuit board section 226B (positioned in the second cavity 222) are physically separate sections that are electrically and/or mechanically coupled to each other. In certain instances, the first circuit board section 226A is a flexible circuit while the second circuit board section 226B is a rigid circuit board. Portions of either or both of the first circuit board section 226A and the second circuit board section 226B can extend through the fourth housing section 212 when the IMD 200 is assembled.

The first circuit board section 226A includes an aperture 234 through which a conductor 236 is passed or inserted into. The conductor 236 can be part of the first electrode 216 or connected to the first electrode 216. The aperture 234 includes a conductive portion such that an electrical signal from the first electrode 216 can be passed from the first electrode 216 to the conductive portion and then to a conductive trace in the first circuit board section 226A. The first circuit board section 226A can include conductors 236 at a proximal end of the first circuit board section 226A to couple to respective conductors of the second circuit board section 226B. For example, one set of conductors can pass electrical signals from the first electrode 216 and another set of conductors can pass electrical signals from the antenna 228.

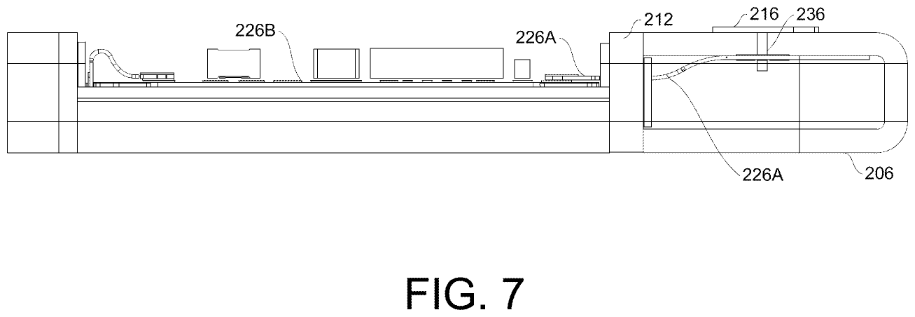

FIG. 7 shows a side view of the IMD 200. In this view, the conductor 236 is shown extending from the first electrode 216 through the first housing section 206 and through the first circuit board section 226A (e.g., through the aperture 234 shown in FIG. 6).

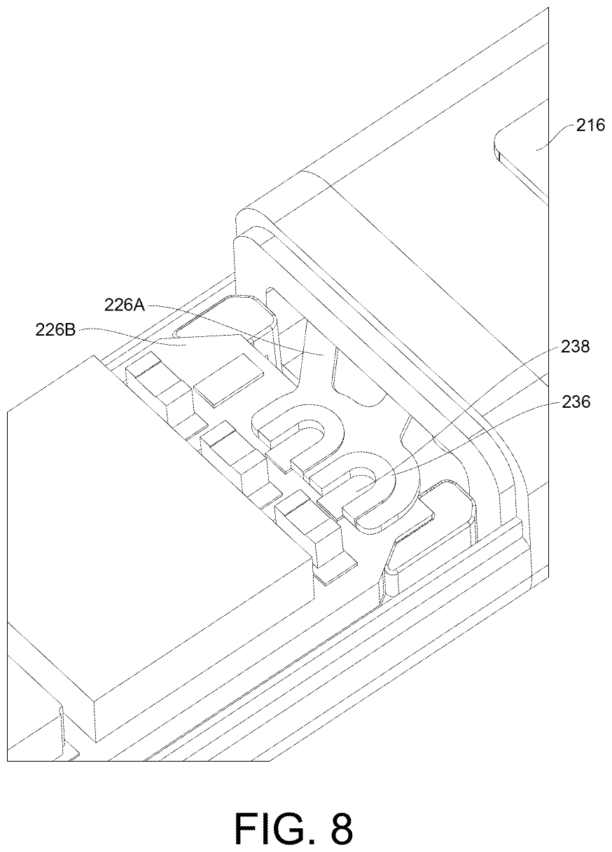

FIG. 8 shows a zoomed-in view of the connection between the first circuit board section 226A and the second circuit board section 226B. In this example, the second circuit board section 226B includes conductors 238 (e.g., conductive bond pads) that electrically and mechanically couple to the conductors 236 of the first circuit board section 226A.

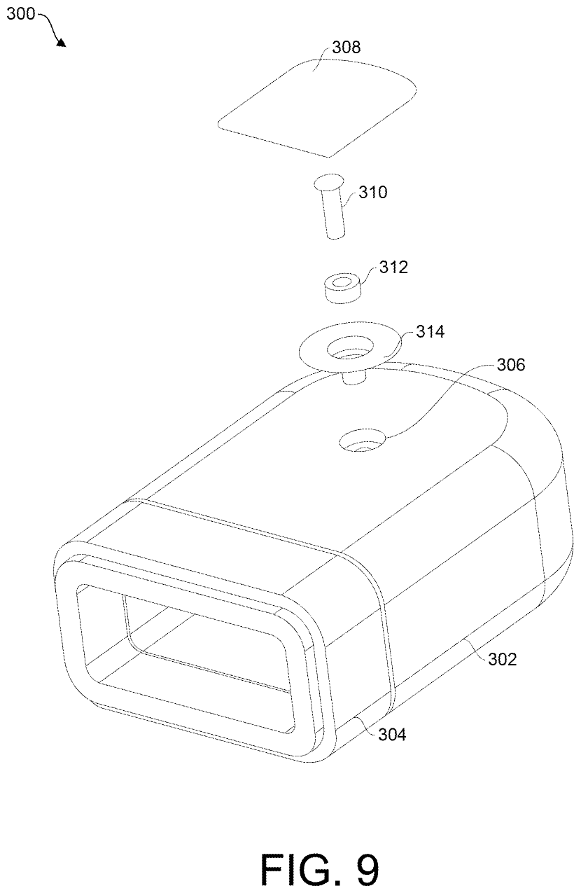

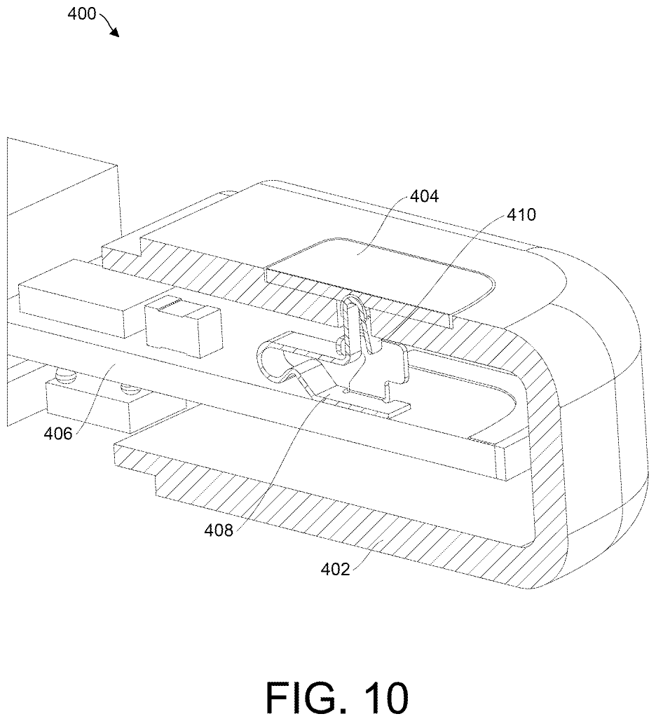

FIGS. 9 and 10 show different views of other example electrical connections between an electrode and a first circuit board section. The approaches shown in FIGS. 9 and 10 can be used with the IMD 200.

In FIG. 9, only a portion of an IMD 300 is shown in an exploded view. The IMD 300 includes a first housing section 302 and a second housing section 304 (e.g., a coupler) connected together and forming an internal cavity or enclosure. The first housing section 302 can comprise a ceramic material. An aperture 306 is formed in the first housing section 302.

The IMD 300 also includes an electrode assembly that hermetically seals the aperture 306. The electrode assembly includes an electrode 308, a conductor 310 (e.g., conductive pin), and a ring 312. When assembled, the electrode 308 is attached (e.g., adhered, brazed, deposited) to an outer surface. In FIG. 9, the metallization of the first housing section 302 is represented by component associated with reference number 314. The conductor 310 is part of or coupled to the electrode 308 and extends through the aperture 306 in the first housing section 302. The ring 312 represents a brazed joint that helps provide a hermetic seal. The ring 312 can comprise a material such as gold or palladium, which can be brazed. The conductor 310 can then be coupled to a circuit board (such as those described above) such that the conductor 310 can transmit signals from the electrode 308 and through the first housing section 302 to the circuit board and ultimately to an electrical component such as a microprocessor.

FIG. 10 shows an IMD 400 with a first housing section 402 forming an internal cavity or enclosure. The first housing section 402 can comprise a ceramic material. The IMD 400 includes an electrode 404 coupled to the first housing section 402 and also includes a circuit board 406. Positioned between the first housing section 402 and the circuit board 406 is a spring contact assembly that includes a spring-loaded base 408 and a conductive contactor 410. When the IMD 400 is assembled, the spring-loaded base 408 forces the conductive contactor 410 to contact the electrode 404 (or an intermediate conductive component such as a pin) so that an electrical connection is maintained between the electrode 404 and the circuit board 406. With the electrical connection, electrical signals can be transmitted from the electrode 404 through an aperture in the first housing section 402 to the circuit board 406 and ultimately to an electrical component such as a microprocessor. The electrode 404 can be coupled to the first housing section 402 to create a hermetic seal. In examples such as that shown in FIG. 10, the circuit board 406 can be a rigid circuit board so that the force from the spring-loaded base 408 is easier to maintain compared to a flexible circuit.

### Methods

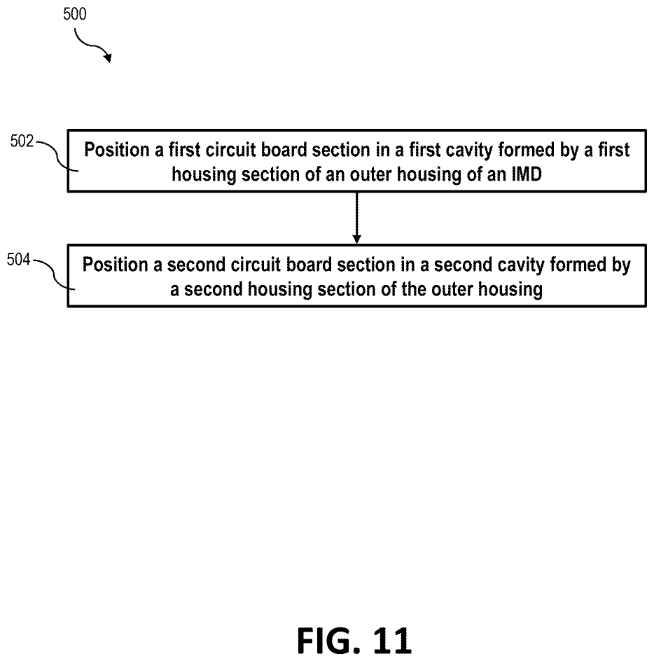

FIG. 11 shows a block diagram of a method 500 of manufacturing the IMDs described above. The method 500 includes positioning a first circuit board section in a first cavity formed by a first housing section of an outer housing for an IMD (block 502 in FIG. 11). The first housing section comprises a ceramic material, and the first circuit board section includes an antenna or is coupled to an antenna. The method 500 further includes positioning a second circuit board section in a second cavity formed by a second housing section of the outer housing for the IMD (block 504 in FIG. 11). The first housing section and the second housing section are coupled together to form a combined cavity in which both the first circuit board section and the second circuit board section are enclosed in.

Various modifications and additions can be made to the exemplary instances discussed without departing from the scope of the disclosed subject matter. For example, while the instances described above refer to particular features, the scope of this disclosure also includes instances having different combinations of features and instances that do not include all of the described features. Accordingly, the scope of the disclosed subject matter is intended to embrace all such alternatives, modifications, and variations as fall within the scope of the claims, together with all equivalents thereof.

We claim:

1. A medical device comprising:
a first housing section including a first cavity and comprising a ceramic material;
a second housing section coupled to the first housing section and including a second cavity;
a first circuit board section positioned within the first cavity;
a second circuit board section positioned within the second cavity; and
an antenna coupled to the first circuit board section and positioned within the medical device to transmit signals through the ceramic material.

2. The medical device of claim 1, wherein the first circuit board section and the second circuit board section comprise a rigid circuit board or a flexible circuit.

3. The medical device of claim 1, wherein the first circuit board section is a flexible circuit board and the second circuit board section is a rigid circuit board.

4. The medical device of claim 1, wherein the first circuit board section and the second circuit board section form a single, continuous circuit board that extends through the first cavity and the second cavity.

5. The medical device of claim 1, wherein the first circuit board section and the section circuit board section are separate circuit boards that are electrically coupled to each other.

6. The medical device of claim 1, wherein the medical device does not include an electrical feed-through positioned between the first housing section and the second housing section.

7. The medical device of claim 6, wherein separate traces embedded within the first circuit board section and the second circuit board section transmit electrical signals from the first housing section to the second housing section.

8. The medical device of claim 1, further comprising:
a first electrode coupled to the first housing section;
a third housing section housing a battery; and
a second electrode coupled to the third housing section.

9. The medical device of claim 8, wherein a pin or spring contact is electrically and mechanically coupled between the first electrode and the first circuit board section.

10. The medical device of claim 9, wherein the pin or the spring contact extends through an aperture in the first housing section.

11. The medical device of claim 10, wherein first electrode or the first pin hermetically seals the aperture.

12. The medical device of claim 1, further comprising:
a third housing section coupled between the first housing section and the second housing section, wherein the third housing section is brazed to the first housing section.

13. The medical device of claim 12, wherein the third housing section is welded to the second housing section.

14. The medical device of claim 1, wherein the antenna is positioned within the first cavity.

15. The medical device of claim 1, wherein the ceramic material comprises zirconia.

16. The medical device of claim 15, wherein the first housing section comprises alumina.

17. A method for manufacturing an implantable medical device (IMD), the method comprising:
positioning a first circuit board section in a first cavity formed by a first housing section of an outer housing for the IMD, wherein the first housing section comprises a ceramic material;
positioning an antenna in the first housing section and coupling the antenna to traces in the first circuit board; and
positioning a second circuit board section in a second cavity formed by a second housing section of the outer housing for the IMD.

18. The method of claim 17, further comprising:
brazing the first housing section to a third housing section; and
welding the second housing section to the third housing section.

19. The method of claim 17, further comprising: hermetically sealing an aperture in the first housing section with one or more components of an electrode assembly.

20. The method of claim 19, further comprising: inserting a conductive pin through the aperture.
